(19) United States
(12) Patent Application Publication
(10) Pub. No.: US 2008/0318918 A1
(43) Pub. Date: Dec. 25, 2008

(54) Methods for Treatment and Prevention of Otitis Media Using Nonionic Surfactants to Facilitate Transmembrane Drug Delivery into the Middle Ear

(76) Inventors: William R. Campbell, Jamestown, NC (US); Neil E. Paulsen, John's Island, SC (US); Roland H. Johnson, Lexington, NC (US)

Correspondence Address: DLA PIPER LLP (US), 4365 EXECUTIVE DRIVE, SUITE 1100, SAN DIEGO, CA, 92121-2133, US

(21) Appl. No.: 12/066,488

(22) PCT Filed: Aug. 25, 2006
(86) PCT No.: PCT/US06/33598

§ 371 Date: Aug. 1, 2008

Related U.S. Application Data
(60) Provisional application No. 60/720,536, filed on Sep. 26, 2005.

(51) Int. Cl. A61K 31/43; A61K 47/10; A61K 31/496; A61K 31/5383; A61K 31/42; A61K 31/522; A61P 31/00

(52) U.S. Cl. 514/194; 514/772; 514/253.08; 514/230.2; 514/378

(57) ABSTRACT

Methods for treating and preventing middle ear infections by transmembrane administration of medicament-containing transmembrane carrier compositions comprising a nonionic polymer surfactant, such as an alkyl aryl polyether alcohol (e.g., tyloxapol) to the tympanic membrane. The medicaments delivered according to the methods of the invention include antibiotic, antiviral, anti-fungal and anti-inflammatory agents that are useful in treatment and/or prophylaxis of middle ear infections and their sequelae.

### FIELD OF THE INVENTION

The present invention relates to non-invasive methods for treating otitis media (middle ear infection). More particularly, the invention relates to methods for administering medicament useful in treating otitis media to the middle ear by delivery thereof across the tympanic membrane (eardrum).

### BACKGROUND

Millions of children are affected each year with otitis media; i.e., infection of the middle ear. Although adults are also susceptible to middle ear infections, children are particularly at risk, because their relatively short auditory canals can more easily be closed by inflammation. Fluid can then become trapped behind the tympanic membrane (eardrum), which can cause severe pain as well as provide microbes with an inviting environment in which to reproduce.

The tympanic membrane is a formidable barrier against introduction of drugs into the middle ear, and so antibiotics prescribed to treat middle ear infections are nearly always taken orally. However, a variety of bacteria and viruses can be responsible for causing middle ear infections, and it is frequently not possible to distinguish which is the cause of a particular infection, or whether it is susceptible to treatment with oral antibiotics. Further, the impact of orally administered antibiotics on the middle ear may be diluted by the systemic distribution of the drug, which may also place the patient at risk for side effects associated with systemic delivery (e.g., yeast infections in female patients).

Children who suffer from repeated infections may require surgery to relieve the fluid pressure on the tympanic membrane. In more severe cases, drainage tubes may be placed within the tympanic membrane. The tubes themselves don't prevent reoccurrences of infection (to the contrary, they can serve as conduits for entry of additional pathogens into the middle ear), but they can relieve pressure and reduce the extent to which fluid becomes trapped behind the eardrum.

The tubes also offer a potential conduit for antibiotics to be introduced directly into the middle ear; e.g., by applying antibiotic drops and allowing them to flow into the drainage tube. However, this method is both invasive and painful, suggesting a strong need for an alternative route for introducing antibiotics into the middle ear.

### SUMMARY OF THE INVENTION

The invention provides methods for treating and preventing otitis media through administration of medicaments useful in prophylaxis or treatment of middle ear infections and their sequelae in a transmembrane carrier composition. The invention derives from the surprising discovery that, in a carrier comprised of one or more nonionic polymer surfactants, medicaments can be delivered across an intact tympanic membrane; i.e., one without tears (e.g., from bursting under pressure) or punctures (e.g., from insertion of tubes or injection).

According to the invention, the medicament is supplied as an active ingredient of a transmembrane carrier composition applied to the ear so as to put the composition into contact with an intact tympanic membrane (eardrum). The transmembrane carrier composition is further comprised of one or more nonionic polymer surfactants, such as a polymer segment or block copolymer, provided in a pharmaceutically acceptable composition.

Preferred medicaments are those useful in the treatment or prevention of otitis media (middle ear infection) and its sequelae. The invention is particularly well-suited to the delivery of medicaments such as antibiotics or anti-viral agents (depending on the source of the infection present), anti-fungal agents, and anti-inflammatory agents or other painkillers. For prevention of chronically recurring middle ear infections, the methods of the invention may also be utilized between active infections to deliver prophylactic agents to the middle ear.

The summary of the invention described above is not limiting and other features and advantages of the invention will be apparent from the following detailed description of the preferred embodiments, as well as from the claims.

### DETAILED DESCRIPTION OF THE INVENTION

### A. Carriers For Use in Transmembrane Treatment of Otitis Media

Surprisingly, it has been discovered that nonionic polymer surfactants, when applied to the tympanic membrane, can facilitate the transport of a medicament across the membrane and into the middle ear. To this end, a nonionic polymer surfactant (e.g., a polymer segment or block copolymer) is provided in a pharmaceutically acceptable composition comprising the medicament. Nonionic polymer surfactants are known in the art (see, e.g., Non-ionic Surfactants, Schick, ed. (Dekker, N.Y., 1967)). A number of such compounds are commercially available under such generic trade names as octoxynol 9 (Triton™ X-100) and its heptamer tyloxapol (Triton™ WR-1339), meroxapol, poloxamers (such as Pluronic™ RTM, F68 and F108), polyxamines such as (Tetronics™ 908, which is a tetrafunctional block copolymer derived from sequential addition of propylene oxide and ethylene oxide to ethylenediamine), dialkylesters of sodium sulfosuccinic acid, such as Aerosol OT™, which is a dioctyl ester of sodium sulfosuccinic acid, Duponol™ P (a sodium lauryl sulfate), Triton™ X-200 (an alkyl aryl polyether sulfonate), polysorbate 20, polysorbate 60, and polysorbate 80 (polyoxyethylene sorbitan fatty acid esters), polyethoxylated castor oils, such as Cremaphor™ EL, and polyethoxylated hydrogenated castor oils, such as HCO-40. Most preferred for use in the invention are polymer surfactants of the alkyl aryl polyether alcohol type; e.g., tyloxapol, and others that have been used, for example, as exogenous pulmonary surfactants, such as exogenous bovine surfactant (Survanta™ beractant), although non-peptidic surfactants (e.g., tyloxapol) are most preferred.

The nonionic surfactant component of the transmembrane carrier composition is present in a range from 0.01% to 10% w/w, preferably from 0.01 to 0.5% w/w, and most preferably from 0.05% to 0.2%, although the exact formulation will vary depending on the presence and amounts of excipients, preservatives, water, pH modulators, and the like included therein. The surfactant is most preferably a liquid at room temperature.

In addition to the nonionic surfactant and medicament, the transmembrane compositions of the invention may contain conventional pharmaceutical excipients and preservatives. In this respect, ‘preservative’ refers to an ingredient added to the transmembrane carrier composition that prevents microbes from substantially growing and multiplying in the formulation. Preferred preservatives include those that are water-soluble and can function as an antimicrobial, such as a benzethonium salt; e.g., benzethonium chloride.

In general, the amount of the preservative ingredient will range from about 0.005-2.0%. Buffers or acids will be added as necessary to adjust the pH of the composition to the preferred range of 3-6, most preferably 4.5 pH. Other preservatives and excipients that may be present in the transmembrane carrier compositions include alkanolamine chloride, sulfate, phosphate, salts of benzoic acid, acetic acid, salicyclic acid, oxalic acid phthalic acid, gluconic acid, 1-naphthalenesulfonic acid, 2-naphthalenesulfonic acid, tartaric acid, maleic acid, malonic acid, succinic acid, fumaric acid, propionic acid, ascorbic acid, mandelic acid, malic acid, citric acid, triethanolammonium chloride, triethanolammonium dihydrogen phosphate, triethanolammonium sulfate, sodium benzoate, potassium benzoate, ammonium benzoate, sodium acetate, potassium acetate, ammonium acetate, sodium salicylate, potassium salicylate, ammonium salicylate, sodium oxalate, potassium oxalate, ammonium oxalate, sodium phthalate, potassium phthalate, ammonium phthalate, sodium gluconate, potassium gluconate, ammonium gluconate, ammonium 1-naphthalenesulfonate, potassium 2-naphthalenesulfonate, ammonium 2-naphthalenesulfonate, sodium 2-naphthalenesulfonate, potassium tartarate, sodium maleate, potassium maleate, sodium malonate, sodium succinate, sodium fumarate, sodium propionate, triethanolammonium propionate, sodium ascorbate, triethanolammonium ascorbate, potassium ascorbate, sodium mandelate, sodium malate, sodium citrate, potassium citrate, and triethanolammonium citrate. Chelating agents may also be utilized; e.g., disodium (“EDTA”); edetate trisodium, edetate tetrasodium, or diethyleneamine pentaacetate.

The composition may also contain other active ingredients, such as anti-inflammatories, analgesics, and steroidal compounds (e.g., hydrocortisone, dexamethasone). Those of ordinary skill in the art will be able to identify suitable compounds and dosages thereof for use in treating pain or inflammation associated with otitis media, such as 0.01-0.5% dexamethasone (e.g., dexamethasone alcohol (preferred), dexamethasone acetate or dexamethasone phosphate).

The compositions are preferably administered with the transmembrane carrier composition itself as a carrier, but in various embodiments the transmembrane carrier may be administered in a carrier gel or other suitable carrier. Buffers or acids; e.g., sodium hydroxide or hydrochloric acid, may be added for adjustment of pH.

### B. Useful Medicaments for Treatment and Prophylaxis of Otitis Media

By “medicament” is meant any biologically active compound useful in the treatment and/or prevention of middle ear infections and their sequelae, as well as associated pain and inflammation. In this respect, therefore, particularly preferred medicaments are antibiotics useful in the treatment or prevention of middle ear infections in mammals, especially humans. Depending on the severity of the infection and its cause, such antibiotics include, without limitation, amoxicillin (and other penicillins), ciprofloxacin (and other quinolone antibiotics, such as ofloxacin), clavulanate (and other beta-lactamase inhibitors), cefaclor (and other cephalosporins, such as cefixime), azithromycin (and other macrolide antibiotics, such as clarithromycin), and sulfisoxazole (as well as other sulfa drugs, such as sulfamethoxazole). Of the antibiotics useful in the invention, ciprofloxacin is presently preferred.

Sulfisoxazole and amoxicillin are the principal antibiotics that are also accepted for use in prophylaxis of recurring middle ear infections. Broad spectrum antibiotics such as amoxicillin and ciprofloxacin are especially preferred for use in treating middle ear infections, especially in persons in whom an antibiotic-resistant infection is suspected.

Useful anti-inflammatory compounds for co-administration or use independent of antibiotic therapy include those that are sometimes less effective or well-tolerated in oral administration; e.g., non-steroidal anti-inflammatory compounds, such as naproxen, ketoprofen, celecoxib and indomethacin. Anti-viral compounds, such as acyclovir, may be administered in lieu of, or as an adjunct to, antibiotic compounds when clinically indicated, as may anti-fungal compositions. Other medicaments for use in the treating and preventing middle ear infections and their sequelae may also be administered by application of the transmembrane carrier compositions of the invention to the tympanic membrane.

In some embodiments, the transmembrane carrier compositions of the present invention contain more than one medicament. For example, CLAMOXYL® and AUGMENTIN® are both combination agent compositions for oral administration that are commonly prescribed for treatment of otitis media. Each composition contains two active antibiotic ingredients, amoxicillin and clavulanate. Transmembrane carrier compositions providing such multiple agents are particularly preferred for use in appropriate indications.

Overall, the medicament is present in whatever concentration is desirable to treat the condition presented. Generally, concentrations of between 0.1 and 10% w/w will be useful, with most useful concentrations falling within the range of 0.2 to 0.5% w/w; i.e., 0.3% to 0.4% w/w will be a typical choice.

### C. Methods for Treating Otitis Media Using the Transmembrane Carriers of the Invention

Although the invention shall not be limited by any theory as to the mechanism of action for such delivery, it is presently believed that the nonionic polymer surfactants present in the transmembrane compositions of the invention modify the porosity and therefore permeability of the tympanic membrane by a magnitude sufficient to permit the medicament to pass into the membrane.

To this end, a transmembrane composition of the invention is delivered, by transmembrane administration, into the middle ear. By “transmembrane administration” is meant that application of a transmembrane carrier composition including a medicament to the outer ear side of the tympanic membrane results in delivery of the medicament to the middle ear. Thus, the invention provides methods for preventing and/or treating infections of the middle ear and their sequelae by transmembrane administration of a medicament to the tympanic membrane of the affected individual.

Transmembrane administration is achieved via, for example, applying the transmembrane carrier composition of the invention to the ear so as to bring the composition into contact with the outer surface of the tympanic membrane via any medically acceptable means for application of a pharmaceutical composition to the tympanic membrane; e.g., by applying the carrier composition to the membrane by insertion of a needleless syringe or dropper into the auditory canal. Care will be taken not to pierce or puncture the intact tympanic membrane.

Administration is repeated as required to achieve the therapeutically effective dosage level for the antibiotic compound and/or other medicament(s) given. Pain may be treated by administration in the same general manner of pain killing and/or anti-inflammatory containing transmembrane carrier compositions of the invention.

Based on current protocols utilized to introduce antibiotics into the middle ear through an in-situ tympanic drainage tube, a suitable regimen of dosing with the exemplary formulation described in Example 1 below (having 0.3% w/w of antibiotic) would be 5 drops/twice a day for a child under age 12, and 10 drops/twice a day for a child of age 12 or older.

Prophylactic treatment against recurrence of a middle ear infection may be provided in the same manner, utilizing a transmembrane carrier composition of the invention containing a prophylactically effective antibiotic or other medicament.

Those of ordinary skill in the art will be familiar with, and readily able to select, dosing regimens suitable for following to treat a particular infection. The dosing regimen selected will be in accord with established clinical protocols for delivery and use of the particular carrier and medicaments provided according to the invention.

The invention having been fully described, its practice is illustrated by the examples below. The invention shall not, however, be limited by the examples, but shall instead be defined in scope by the appended claims.

### EXAMPLE 1

### Exemplary Formulation

Following is an example of the a transmembrane carrier composition of the present invention containing ciprofloxacin and tylopaxol, as follows (all concentrations are % w/w): Ciprofloxacin HCl, 0.35 (equivalent to 0.3% ciprofloxacin base); monohydrate dexamethasone alcohol 0.1 (equivalent to 1 mg dexamethasone base); hydroxyethyl cellulose 0.2; benzalkonium chloride 0.01; sodium acetate 0.03; (trihydrate) acetic acid (as a buffer) 0.04; sodium chloride 0.25; EDTA 0.01; tyloxapol 0.05; glycerin 1.5; boric acid 0.6; NaOH/HCl as needed to adjust pH to 4.5+−0.2; purified water to form an aqueous composition.

The composition is sterilized and placed in a pharmaceutically acceptable container until use.

### EXAMPLE 2

### Animal (Chinchilla) Model of Otitis Media

Chinchilla langer is ideally suited as an animal species for studying the efficacy of treatment for otitis media in humans. Chinchillas are small, have auditory capabilities quite similar to those of humans, have a cochlea with membranous architecture similar to the human cochlea, do not manifest presbycusis in long-term studies, and lack susceptibility to naturally occurring middle ear infections, which are common to the guinea pig and rabbit, See, e.g., Hajek D M, Yuan Z, Quartey M K, Giebink G S., Otitis Media: The Chinchilla Model, in: Zak O, Sande M, editors, Handbook of Animal Models of Infection, San Diego, Calif.: Academic Press (1999), at pages 389-403, the contents of which are incorporated herein by reference to illustrate the nature and acceptance in the art of this animal model.

To establish and evaluate the animal model, each chinchilla was inoculated with Haemophilus influenzae directly into the middle ear of each ear by transbullar injection at a concentration of 100 cfu in a volume of 0.2 mL. Each chinchilla was given an otoscope ear exam prior to being placed on study. Dosing with a composition of the invention or control oral amoxicillin began approximately 48 hours after the bacterial inoculation. All animals were administered Buprenorphine 0.05 mg/kg twice a day subcutaneously for analgesia for the duration of the study.

At the end of the dosing period (8 days after bacterial inoculation), each animal was euthanized, their ear canals washed with saline, and examined. In particular, samples from the middle ear from each chinchilla were collected. One ear sample was cultured overnight per laboratory procedures. Approximately 24 hours after the samples plated out, they were counted and the colony forming units (cfu) recorded.

### EXAMPLE 3

### Treatment of Otitis Media in Chinchilla Model

The formulation described in Example 1 was administered orally by gavage to three chinchillas twice per day for 6 days, approximately 8 hours apart. 2, 4 or 6 drops of the formulation were administered to two groups of three chinchillas each as a maximal feasible dose for these animals. The animals were examined, and samples were taken from the middle ear of each as described in Example 2. The following results were obtained:

| | Concentration (ng/ml) of | | | Number ears |
|---|---|---|---|---|
| | ciprofloxacin | | | infected/total |
| Group | detected in middle ear | | | number ears |
| 1 | None | | | 7 of 10 |
| (Untreated) | | | | |
| 2 | Animal | Ear | | 0 of 10 |
| (Treated) | | Concentration | | |
| | 1016 | L | 3096.74 | |
| | | R | 246.86 | |
| | 1017 | L | 324.07 | |
| | | R | 264.82 | |
| | 1018 | L | 612.13 | |
| | | R | 15.95 | |
| | 1019 | L | 1012.09 | |
| | | R | 1701.02 | |
| | 1020 | L | 662.51 | |
| | | R | 37.55 | |

These results demonstrate the efficacy of the present invention in treating middle ear infection in a relevant animal model.

The invention illustratively described herein may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other documents.

The inventions illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. Other embodiments are set forth within the following claims.

In addition, where features or aspects of the invention are described in terms of Markush groups, those skilled in the art will recognize that the invention is also thereby described in terms of any individual member or subgroup of members of the Markush group.

That which is claimed is:

1. A method for treating or preventing a middle ear infection and sequelae thereof by transmembrane administration of a medicament thereto, said method comprising:
applying a transmembrane carrier composition to the outer surface of the tympanic membrane, said transmembrane carrier composition comprising a medicament useful in treating or preventing infections of the middle ear and sequelae thereof.

2. The method according to claim 1, wherein the transmembrane carrier is a nonionic polymer surfactant.

3. The method according to claim 2, wherein the nonionic polymer surfactant is an alkyl aryl polyether alcohol.

4. The method according to claim 1, wherein said medicament is an antibiotic.

5. The method according to claim 4, wherein the antibiotic is selected from the group consisting of quinolone antibiotics, penicillin antibiotics, macrolide antibiotics, cephalosporin antibiotics, sulfa antibiotics, and beta-lactamase inhibitors.

6. The method according to claim 4, wherein said antibiotic comprises ciprofloxacin, and is administered to treat or prevent a middle ear infection.

7. The method according to claim 4, wherein said antibiotic comprises ofloxacin, and is administered to treat or prevent a middle ear infection.

8. The method according to claim 4, wherein said antibiotic comprises sulfisoxazole, and is administered to treat or prevent a middle ear infection.

9. The method according to claim 4, wherein said antibiotic comprises amoxicillin, and is administered to treat or prevent a middle ear infection.

10. The method according to claim 4, wherein the antibiotic is provided in a concentration of 0.1% to 10% w/w of the composition.

11. The method according to claims 4, wherein the antibiotic is provided in a concentration of 0.3% w/w of the composition.

12. The method according to claim 2, wherein the nonionic polymer surfactant is provided in a concentration of about 0.01 to 10% w/w.

13. The method according to claim 2, wherein the nonionic polymer surfactant is provided in a concentration of about 0.05% to about 0.2% v/v.

14. The method according to claim 1, wherein said medicament is an anti-viral agent.

15. The method according to claim 14, wherein the anti-viral agent is acyclovir.

16. The method according to claim 2, wherein the alkyl aryl polyether alcohol is tyloxapol.

17. The method according to claim 1, wherein the transmembrane carrier composition is applied to the tympanic membrane during an acute phase of middle ear infection.
